(19) United States
(12) Patent Application Publication
(10) Pub. No.: US 2022/0228193 A1
(43) Pub. Date: Jul. 21, 2022

(54) METHODS TO DETECT CELLS LATENTLY INFECTED WITH HIV

(71) Applicant: Oxford University Innovation Limited, Oxford (GB)

(72) Inventors: Matthew Lee JONES, Oxford (Oxfordshire) (GB); Alexander John FRATER, Oxford (Oxfordshire) (GB)

(21) Appl. No.: 16/629,582

(22) PCT Filed: Jul. 10, 2018
(86) PCT No.: PCT/GB2018/051959

§ 371 Date: Jan. 9, 2020

(30) Foreign Application Priority Data
Jul. 10, 2017 (GB) 1711066.9

(51) Int. Cl. C12Q 1/686

(52) U.S. Cl. CPC C12Q 1/686; G01N 2800/26

(57) ABSTRACT

The present invention provides a method of identifying a cell latently infected with HIV, wherein the method comprises: providing a sample of cells; encapsulating individual cells in droplets; screening for the presence of HIV derived DNA in the genomic DNA of encapsulated cells; and identifying, and optionally isolating, cells containing latent HIV derived DNA.

### CROSS-REFERENCE TO RELATED APPLICATIONS

This application is the National Stage of International Application No. PCT/GB2018/051959, filed Jul. 10, 2018, which claims the priority to GB 1711066.9, filed Jul. 10, 2017, which are entirely incorporated herein by reference.

The present invention relates to methods for detecting and analysing cells latently infected with viral DNA, in particular HIV-1 DNA, and to uses of these methods.

### BACKGROUND

In 2014 more than 37 million people were living with HIV-1 infection (http://www.unaids.org). Optimal patient outcomes are achieved by initiating combination antiretroviral therapy (ART) as soon as infection is diagnosed. ART reduces plasma virus levels to below the clinical detection limit (20-50 copies of HIV-1 RNA/ml) and halts disease progression. Although ART effectively suppresses viremia, it is not curative, and viremia usually rebounds upon ART cessation. Therefore, lifelong ART treatment is required. Providing lifelong treatment for all infected individuals poses a major economic and logistical challenge. 15 million people currently receive ART. The tolerability of ART regimens has improved dramatically, but long-term drug toxicity is a concern. Other problems include the emergence of resistance with suboptimal treatment and the stigma associated with the infection. For these reasons, there is great current interest in a cure.

The principal barrier to cure is the existence of a stable reservoir of latent HIV-1 in resting CD4+ T cells. The reservoir persists even in patients on long-term ART who have no detectable viremia. The cells comprising this reservoir have a memory phenotype. Direct measurements of the latent reservoir in patients on ART show a very slow decay rate (estimated t_{1/2} of 3.7 years). At this rate, eradication of a reservoir of 10⁶ cells would require 73 years, making cure unlikely even with lifelong ART.

Reactivation of these latent viruses appears to be largely stochastic but can be triggered by a change in the activation status of the cell. In the absence of ART, these reactivated cells can serve to re-seed productive infection leading to viral rebound. The viral reservoir therefore is seen as the key contributing factor in the return of viraemia and disease progression on treatment cessation. Thus, research toward a cure focuses on eliminating this reservoir.

Reservoir dynamics in the context of ART are currently poorly understood. CD4+ T cells, in particular TCM (central memory) are thought to be the largest reservoir of latent infection. Even in the TCM population, as few as one in a million cells may be infected, with no current technology available to enrich and characterise such a rare population. Reservoir studies have thus far proved difficult, with no unique biomarkers identified it has not been possible to enrich or accurately quantify this population.

Current measures of latency are not very accurate. The Viral Outgrowth Assay (VOA) has been regarded as the ‘gold standard’ for measuring the HIV reservoir as it only quantifies replication-competent virus. The assay uses either polyadenylated HIV-1 RT-PCR or p24 ELISA to measure the ability of primary CD4 T-cells to produce virions. The reservoir is however underestimated, as a single round of activation is not sufficient to cause all replication-competent viruses to produce virions. Molecular qPCR assays targeting multiple intracellular DNA species (integrated, 2-LTR circles, non-integrated) are in common use. These assays are quick and inexpensive, however are limited by overestimating the size of the reservoir as they only require a short (150-200 bp) region of the HIV-1 genome to be intact. They therefore quantify mutated viral species that would not be able to initiate productive infection in vivo. Quantifying intracellular RNA may also assist in predicting rebound in ART treated patients. Assays quantifying both unspliced and spliced RNA species are now available. The recently developed tat/rev induced Limiting Dilution Assay (TILDA) quantifies cells harbouring viral genomes capable of producing tat/rev multiply spliced RNA (msRNA). This assay has the benefit (over unspliced RNA) of better predicting replication-competence, as many viruses unable to produce rev, and particularly tat transcripts, have been shown to be non replication-competent.

However, there remains a need for a more representative assay. The assays detailed above represent the state of the art for HIV-1 reservoir quantification. All of the assays have shortcomings that result in either an under- or over-representation of the true size of the reservoir. Recent studies have demonstrated that DNA sequence analysis is a crucial factor when determining replication-competence (Ho, Y.-C. et al. Cell 155, 540-51 (2013)). In a recent publication it was demonstrated that >93% of cells containing integrated HIV-1 DNA carry large insertions or deletions (Bruner, K. M. et al. Nat. Med. 22, 1043-9 (2016)). Viruses containing these mutations are non replication-competent and therefore should not be considered when quantifying the true reservoir.

An aim of the present invention is to provide a method of determining the size of the reservoir of replication-competent latent HIV-1 in a subject. The method may also be used to identify other rare cells in a population based on genomic differences—for example to identify cells infected with any virus with an intracellular latent stage in its life cycle, or to identify cells where the biomarker is in the nucleic acid, and not on the cell surface, for example certain circulating tumour cells.

In an aspect, the present invention provides a method of using genotypic and phenotypic analysis to identify rare cells in a population, wherein the method comprises:
providing a sample of cells;
screening for the presence of cells expressing a particular phenotypic marker;
screening for the presence of cells expressing a particular genotypic marker; and
identifying, and optionally isolating, cells with one or more of the following properties:
   cells expressing the particular phenotypic marker;
   cells expressing the particular genotypic marker;
   cells expressing the particular phenotypic and genotypic marker; and
   cells expressing neither the phenotypic or the genotypic marker.

In another aspect, the present invention provides a method of identifying a cell latently infected with HIV, wherein the method comprises:
providing a sample of cells;
screening for the presence of HIV derived DNA in the genomic DNA of individual cells in the sample; and
identifying cells containing latent HIV derived DNA.

Preferably the sample of cells is obtained from a biological sample obtained from a subject.

The sample cells may be derived from a tissue biopsy, a blood sample, a sample of any other bodily fluid, or indeed any sample from which single cells can be derived. If the sample is whole blood the sample may be processed to isolate peripheral blood mononuclear cells.

The sample may be processed to produce a solution of single cells. The number of cells needed will depend on the frequency of infected cells. Preferably between 4 and 8×10⁶ cells will be sufficient.

The isolated cells may be subjected to an enrichment step to isolate and enrich for CD4⁺ T-cells in the sample. The enrichment step may be antibody based.

The method of the invention may optionally further include the step of obtaining the biological sample from the subject.

The subject may be a human. The subject may have been previously diagnosed with an HIV infection. The subject may be taking antiretroviral therapy.

Preferably the method of the invention is for identifying latent HIV-1 or HIV-2 infection in CD4 T cells or other potential host cells, more preferably latent HIV-1 infection.

In order to screen individual cells in the sample, the cells may be individually encapsulated before analysis. Individual cells may be encapsulated in droplets for further analysis. The droplets may be water in oil droplets. Cells in the droplets may be manipulated to analyse the encapsulated cell. In an embodiment, the individual cells may be encapsulated in a droplet with the reagents needed to detect the presence of HIV derived DNA in the genomic DNA of the cell. The reagents may comprise a Polymerase Chain Reaction (PCR) mix comprising the primers and enzymes necessary to amplify and detect a specific DNA sequence, such as HIV derived DNA, in the genomic DNA of the encapsulated cell. In an alternative embodiment the cell may be encapsulated in a droplet with the reagents need to perform an isothermal DNA amplification reaction, to detect specific DNA, such as HIV derived DNA, in the genomic DNA of the encapsulated cell, the reagents may include appropriate primers and a lysis reagent. In a further embodiment the PCR reagents or the isothermal DNA amplification reagents may be added to a droplet containing a cell after the cell has been encapsulated, preferably after the cell has been lysed, more preferably after both the cell membrane and the nuclear membrane have been lysed. The reagents may be added by injecting them into the droplet, or by fusing the droplet with one or more other droplets, wherein the one or more other droplets contain the reagents to amplify the genomic DNA in the droplet comprising the cell. Where the reagents are added after the cell has been encapsulated the cell may first be lysed before the amplification reagents are added. The cell membrane and the nuclear membrane may be lysed before the amplification reagents are added. In an embodiment of the invention the nuclear membrane is lysed to allow access to the genomic material, preferably the DNA. The conditions used to lyse the nuclear membrane may result in an environment in the droplet that is not conducive to DNA amplification, it may therefore be important to include a step in the method to alter the environment to allow DNA amplification. This may be achieved by including a step to dilute the droplet, for example by fusing the droplet with one or more other the droplets which do not contain cells. The one or more other droplets may contain the reagents need to amplify the genomic DNA. In another embodiment, after the cell membrane and nuclear membrane are lysed in the droplet, an agent used to lyse the cell membrane and/or the nuclear membrane are inactivated. The inactivation may be achieved by heating the droplets or by chemical neutralisation. The amplification reagents may be added after the lysis agents are inactivated. The amplification reagents may be added by injection into the droplet or by fusing the droplet after the cell is lysed with one or more droplets containing the amplication reagents.

In an embodiment the agents used to lyse the cell membrane and/or nuclear membrane in a cell encapsulated in a droplet are (i) proteinase K and (ii) SDS and/or Triton X-100. After lysis has occurred the proteinase K may be heat inactivated before the one or more DNA amplification reagents, in particular the DNA polymerase, are added to the droplet. Triton X-100 is compatible with DNA amplification and can be used as an alternative to SDS in order to negate its inhibitory effect on PCR. Tween 20 may be added to neutralise the effect of SDS, and may be added by droplet fusion or by injection into the droplet.

The method of the invention preferably allows in droplet cell membrane and nuclear membrane lysis and subsequent DNA amplification.

In an embodiment where primers are used, be it for PCR or isothermal amplification, the primers may be labelled with a visual tag, for example a fluorescent tag, which allows the presence of HIV derived DNA to be observed. The fluorescent tag could be on a probe, such as a taqman probe. For example, if PCR or isothermal amplification of target DNA has occurred in a cell in a droplet a fluorescent signal may be observed.

Droplets of the invention may be stabilised by the inclusion of a non-ionic detergent. The non-ionic detergent may be octylphenoxy poly(ethyleneoxy)ethanol, branched, also known as Igepal™ and have the formulae (C₂H₄O)ₙC₁₄H_{22:}

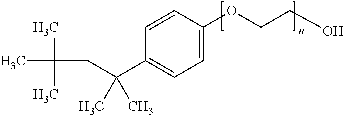

wherein n may on average be between 1 and 20, preferably between 5 and 12. In an embodiment, n is on average 9 or 10 as in Igepal 630.

Where cells are encapsulated in a droplet prior to analysis of the genomic DNA the method may comprise a further screening step to allow the number of droplets containing a cell to be determined—irrespective of whether the cell carries a specific genomic marker, such as latent HIV DNA. This can be done by using a labelled antibody or probe against a phenotypic marker such as a cell surface marker, or by performing a PCR or an isothermal DNA amplification reaction for a human genomic target using labelled primers, to confirm the presence of a cell in a droplet. Where an antibody is used this may be added to the cell population prior to encapsulation of the cell.

The label used in any step in the method of the invention may include any molecule that can be detected by physical, chemical, electromagnetic or other analytical technique. Examples of detectable labels that can be utilised include, but are not limited to, radioisotopes, fluorophores, chromophores, mass labels, electron dense particles, spin labels, molecules that emit chemiluminescence, electrochemically active molecules, enzymes, cofactors, enzyme linked to nucleic acid probes and enzyme substrates.

In an embodiment, the screening step may include counting the number of cells carrying latent HIV. Preferably the screening step comprises determining the number of cells in a sample and determining how many of those cells carry latent HIV. The number of cells in a sample may be determined by using a phenotypic marker, such as a cell surface marker. Preferably the cells are individually encapsulated in droplets.

Without further analysis it may not be possible to say how many of the cells which are identified as carrying HIV actually carry replication competent HIV. In order to assess this, the method of the invention may comprise a further step, namely the step of isolating cells found to carry latent HIV from the rest of the cell population for further analysis. Flow cytometry, magnetic sorting, dielectrophoretic current, standing acoustic waves, optical tweezers or any other suitable method may be used to isolate single cells identified as carrying latent HIV.

In an embodiment a flourescent label is used in the PCR process and cells with a fluorescent intensity above a stated threshold are selected using a dielectrophoretic charge to deflect selected droplets into a sort channel. Preferably the sorting is carried out on a microfluidic chip, preferably the same chip on which the DNA amplification was carried out on.

Cells identified as carrying latent HIV may be sorted into individual wells in a multiwell plate for subsequent analysis, such as by sequencing. Preferably the analysis of the individual cells identified to carry a latent HIV virus is high throughput.

By analysing the cells individually, the replication competency of cells in a population carrying latent HIV can be determined.

The genomic DNA of the isolated cells may then be analysed, preferably on a cell by cell basis, for example by sequence analysis, to determine if the HIV DNA present is replication competent. This may be achieved by sequencing the entire genome, for example by using next generation sequencing, or by sequencing just the HIV DNA, for example by using targeted sequencing methods.

Cells identified as carrying latent HIV may be sorted and collected, once collected a DNA enrichment step may be undertaken on the recovered DNA. The enrichment process may use an RNA bait assay to enrich for DNA of interest. This enrichment process may allow the site of HIV integration to be identified and sequenced. It also allows deep sequencing of the virus to aid in detecting minor variants that may not otherwise be possible.

In a preferred embodiment of the invention, droplets and/or cells may be analysed for both (i) a phenotypic marker—for example, do they express a particular cell surface or intracellular protein which indicates the presence of a cell; and (ii) a genotypic marker—for example, does a particular cell contain a specific target DNA sequence, such as HIV DNA, in the genomic DNA, and optionally is the DNA replication competent.

In an embodiment the method of the invention, or at least some steps of the method of the invention, are carried out using a microfluidic device (sometimes also referred to as a microfluidic chip). Microfluidic devices are well known in the art and comprise a set of micro flow channels etched or molded into a material (typically glass, silicon or polymer such as PDMS—poly-dimethylsiloxane). The flow channels are connected together in order to achieve the desired features of the device, and provide a flow path through which solutions can flow. The device may also include valves to control fluid flow and to isolate fluid selectively in chambers on the device.

The dimensions of the flow channels in a microfluidic device can vary widely. Typically channels are from about 0.1 μm to about 1000 μm in any dimension, sometimes from about 0.1 to about 100 μm, and sometimes from about 0.1 to about 10 μm. Appropriate channel dimensions will depend in part on the nature of the entities being used in the chip. For example, for eukaryotic cells the dimension should be at least sufficient for passage of the cell (e.g., 2-5 times the dimension of the cell). However, for the purpose of restricting movement the dimensions can be on the order of 0.75 times the smallest dimension of a cell.

Reactions, for example nucleic acid amplification or protein/antibody binding, may be allowed to occur in a chamber on the device. For example, PCR reactions can be initiated by heating a chamber (for example, by placing the device on a suitably programmed flat plate thermocycler). In this embodiment the reactions may be, for example, using an antibody to confirm a cell is present, and/or may be using PCR to demonstrate the presence of a cell and/or to demonstrate the presence of a specific target DNA sequence in the genomic DNA in a cell, for example latent HIV in the genomic DNA of the cell.

The results or products of the reactions carried out on the device can be detected using any of a number of different detection strategies. The nature of the signal to be detected will, of course, determine, to a large extent, the type of detector to be used. The detectors can be designed to detect a number of different signal types including, but not limited to, signals from radioisotopes, fluorophores, chromophores, electron dense particles, magnetic particles, spin labels, molecules that emit chemiluminescence, electrochemically active molecules, enzymes, cofactors, enzymes linked to nucleic acid probes and enzyme substrates. Illustrative detection methodologies suitable for use with the present microfluidic devices include, but are not limited to, light scattering, multichannel fluorescence detection, infra-red, UV and visible wavelength absorption, luminescence, differential reflectivity, and confocal laser scanning. Additional detection methods that can be used in certain applications include scintillation proximity assay techniques, radiochemical detection, fluorescence polarization, fluorescence correlation spectroscopy (FCS), time-resolved energy transfer (TRET), fluorescence resonance energy transfer (FRET) and variations such as bioluminescence resonance energy transfer (BRET). Additional detection options include electrical resistance, resistivity, impedance, and voltage sensing.

A detector can include a light source for stimulating a reporter that generates a detectable signal. The type of light source utilized depends in part on the nature of the reporter being activated. Suitable light sources include, but are not limited to, lasers, laser diodes and high intensity lamps. If a laser is utilized, the laser can be utilized to scan across a set of detection sections or a single detection section. Laser diodes can be micro fabricated into the microfluidic device itself. Alternatively, laser diodes can be fabricated into another device that is placed adjacent to the microfluidic device being utilized to conduct a thermal cycling reaction such that the laser light from the diode is directed into the detection section.

Detectors can be micro fabricated within the microfluidic device, or can be a separate element. A number of commercially-available external detectors can be utilized. Many of these are fluorescent detectors because of the ease in preparing fluorescently labelled reagents.

In the present invention preferably the cells are encapsulated in droplets on the microfluidic device. This may be achieved by introducing in one flow channel an aqueous cell suspension and in another flow channel an oil, preferably with a fluoro-surfactant, and controlling the flow such that when the two flow channels meet at a flow focusing junction and water in oil droplets form with no more than one cell per droplet. If other reagents are required in the droplet, for example PCR reagents, antibodies, barcoding beads, these can be added by including in them in the above mentioned aqueous flow, or adding a further third aqueous flow channel containing the reagents upstream of the flow focusing junction.

As well as determining the products of a reaction, for example a nucleic acid amplification reaction or protein/antibody binding, the products of one or more reactions may be used to allow the partitioning of individual cells with specific properties. The cells may be partitioned on the microfluidic device, or the cells may be removed from the device and then separated. The cells may be partitioned into those with a specific phenotypic marker and/or a specific genotypic marker, the genotypic marker may be latent HIV DNA, and those without.

Cells and/or droplets may be sorted or selected on the microfluidic device by using dielectrophoretic current, standing acoustic waves, flow cytometry or optical tweezers.

If droplets and/or cells are removed from the microfluidic device before sorting or separation, then flow cytometry may be used to effect separation. If the cells are to be analysed by flow cytometry (on or off the microfluidic device), and they are encapsulated in a water in oil droplet, they may be further encapsulated in water to create a water in oil in water droplet before further analysis, so they can be used in a flow cytometer. The water in oil in water droplet may be generated on a microfluidic chip. The water in oil in water droplet may be generated before or after the amplification step.

Cells isolated by the method of the invention may be analysed individually without any background from other cells in the sample. The isolated cells may be analysed for proteomic, genomic or transcriptomic traits or differences.

If the genomic DNA of the isolated cells is to be sequenced this may be carried out on or off the microfluidic device.

A utility of the method of the invention may be in clinical research. In an embodiment, the method may provide an accurate measure of the size and nature of the reservoir of latent HIV in a subject. This measure may give an indication of a patient's prognosis, particularly in the context of a proposed treatment interruption. Currently the majority of clinical trials addressing an HIV cure include at least one measure of the reservoir in their protocol. The absence of a gold standard assay in the field has caused different trials to use different reservoir measures. This inconsistency in reporting makes comparison of clinical interventions between trials very difficult as a modification in reservoir size is expected to be a key measure of an intervention's failure or success. As we move into a period in HIV-1 research that is likely to see an increasing number of clinical trials attempting controlled treatment interruptions, it is crucial the field has an accurate measurement of the reservoir. This is necessary both in terms of selecting eligible candidates for potential interruption and also for assessing any detrimental effects interruption may have on reservoir size and diversity.

The method of the invention also has utility in the clinic where HIV-1 reservoir size is expected to be as important a predictor of a patient's prognosis as CD4+ cell count and viral load. This may particularly be the case in patients on effective ART where viremia will usually be undetectable.

The method of the invention may allow the size and/or the nature of the reservoir of replication-competent HIV-1 in a subject to be determined. This may be achieved by calculating how many cells in the total cell population are carrying replication competent HIV DNA.

In a further aspect the invention provides a method of determining the size of the reservoir of cells latently infected with HIV-1 in a subject comprising:
providing a sample of CD4+cells from a subject;
encapsulating individual cells in droplets comprising primers specific for a HIV-1 target;
subjecting the cells to conditions to allow amplification of HIV-1 derived DNA if present in the genome an encapsulated cell;
screening for droplets to identify in which DNA amplification has occurred;
quantifying the number of cells that contain HIV-1 derived DNA;
quantifying the total number of cells;
using the total number of cells and the number of cells that contain

HIV-1 derived DNA to determine the size of the reservoir of cells latently infected with HIV-1 in a subject.

This method may include further steps to allow the reservoir of cells latently infected with HIV-1 to be analysed further to determine how many of the cells are latently infected with a replication-competent HIV-1 virus. This method may include the further step of isolating individual cells/droplets that contain HIV-1 derived DNA and then analysing the HIV-1 derived DNA to determine if it is replication-competent. This may be achieved by full-length proviral sequencing.

The method may include the further step of detecting how many droplets actually contain a cell. This may be achieved by screening for a phenotypic marker in the droplets.

According to an aspect, the present invention provides a method of determining the replication competent viral load of a subject diagnosed with an HIV infection, wherein the method comprises the steps of:
i) obtaining a blood sample from the subject;
ii) enriching for CD4⁺ T cells in the sample;
iii) encapsulating individual CD4⁺ cells in water in oil droplets on a microfluidic device, optionally in the presence of DNA amplification reagents;
iv) lysing the cells in the droplets, preferably lysing the cell membrane and the nuclear membrane, to allow access to the DNA;
v) introducing into the droplet DNA amplification reagents if not already present, the DNA amplification reagents may be introduced by injection into the droplet or droplet fusion;
vi) placing the device in conditions that permit amplification of any HIV DNA in a cell in a droplet;
vii) counting and optionally isolating droplets in which amplification of HIV DNA has occurred; and optionally
viii) analysing the genomic DNA in the isolated droplets to determine if the HIV DNA is replication competent.

The method may include the further step of determining the total number of droplets containing a cell and the total number of droplets with HIV DNA present, from which the HIV load of the subject can be determined. By determining the total number of droplets containing a cell and the total number of droplets with replication competent HIV DNA present, the true risk of the HIV infection rebounding can be determined. This may be used to decide whether or not a subject can be taken off ART.

This method of the invention may further include one or more of the following features:
in step i) the sample may be a tissue sample;
in step ii) the cells may be enriched using a labelled probe, such as an antibody, for a cell surface marker. This marker may then be used to determine how many droplets contain a cell, and thus the total number of cells analysed. The probe may be used to isolate a population of CD4⁺ T cells for further study;
in step iii) the encapsulation of individual cells may be carried out using a fluid focusing microfluidic device;
in step vi) the amplification may be by PCR or isothermal amplification. Preferably the amplification reaction is carried out on the microfluidic device;
the droplets containing cells in which DNA amplification occurred may be separated from the rest of the cells on the microfluidic device, or the droplets may be removed from the device and then sorted, for example by using flow cytometry. The droplets may be converted into water in oil in water droplets, optionally on the same or a different microfluidic device, prior to the isolation of cells in which DNA amplification occurred;
in step viii) the analysing of the HIV DNA to determine if it is replication competent may be undertaken by next generation sequencing or by targeted PCR and sequencing of the PCR products. The sequencing may be carried out on the microfluidic device or off the microfluidic device. An enrichment step may be included to enrich for sequences of interest.

A method of the invention may be used to determine or monitor the efficacy of a particular therapy. By determining the effect that a particular therapy has on the HIV-1 viral load, and in particular the replication competent HIV-1 viral load, the efficacy of the drug can be determined.

The method of the invention has the advantage that is allows cells present in as little as one in a million cells to be detected, isolated and further analysed.

The method of the invention can be used to generate droplets at around 5,000-10,000 droplets/second—1.8 C/hr.

The skilled person will appreciate that all preferred or optional features of the invention may be applied to all aspects and embodiments of the invention.

Embodiments of the present invention will now be described, purely by way of example, with reference to the accompanying drawings, in which:

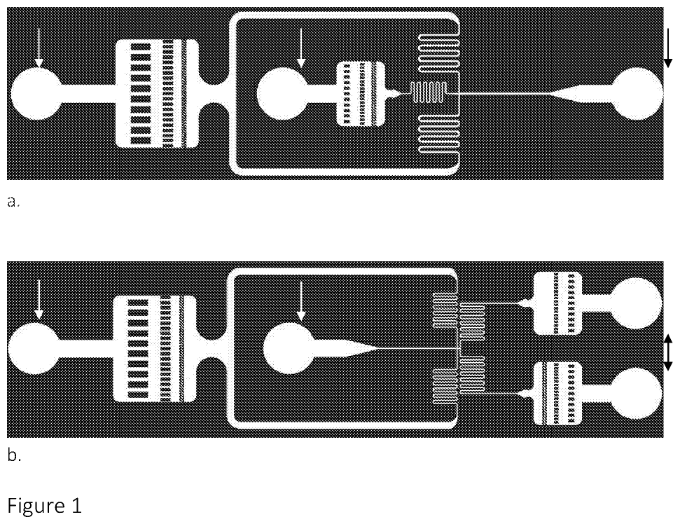

FIG. 1—details the layout of microfluidic devices that can be used to perform the method of the invention. Figure la shows a WO (water in oil) droplet chip, and details the photomask of a PDMS droplet chip used to create WOs detailing sample inlets (red arrows—to the left and right side of the figure) and sample outlet (green arrow in the middle of the figure). FIG. 1b shows a WOW (water in oil in water) droplet chip, and details the photomask of a PDMS droplet chip used to create WOWs detailing sample inlets (red arrows—to the left and right side of the figure) and sample outlet (green arrow—in the middle of the figure).

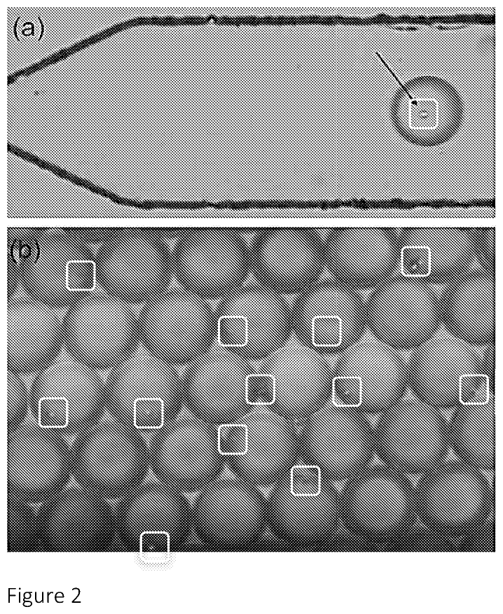

FIG. 2—demonstrates that by using the device of Figure la cell encapsulation occurs. FIG. 2a is an image showing a single cell (inside the square) loaded within a WO. FIG. 2b shows some droplets are occupied by a cell (illustrated by the square) and some droplets are not.

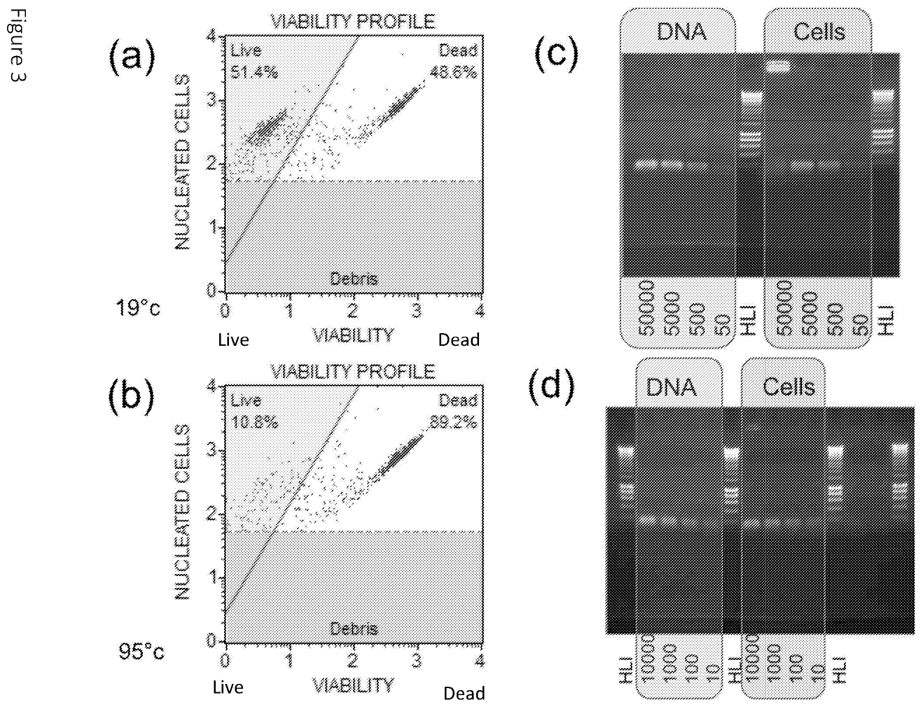

FIG. 3—shows the results of lysis PCR. FIG. 3a shows cell viability after incubation for 3 minutes at 19° C. FIG. 3b shows cell viability after incubation for 3 minutes at 95° C. FIG. 3c shows an electrophoresis gel showing successful PCR amplification of a 150bp product using both extracted 8E5 cell DNA and 8E5 cell lysate at varying concentrations (range 50-50,000). FIG. 3d is a repeat assay of FIG. 3c using a lower concentration series of 8E5 DNA and cells (10 -10,000). 8E5 is a cell line in which the cells are infected with HIV.

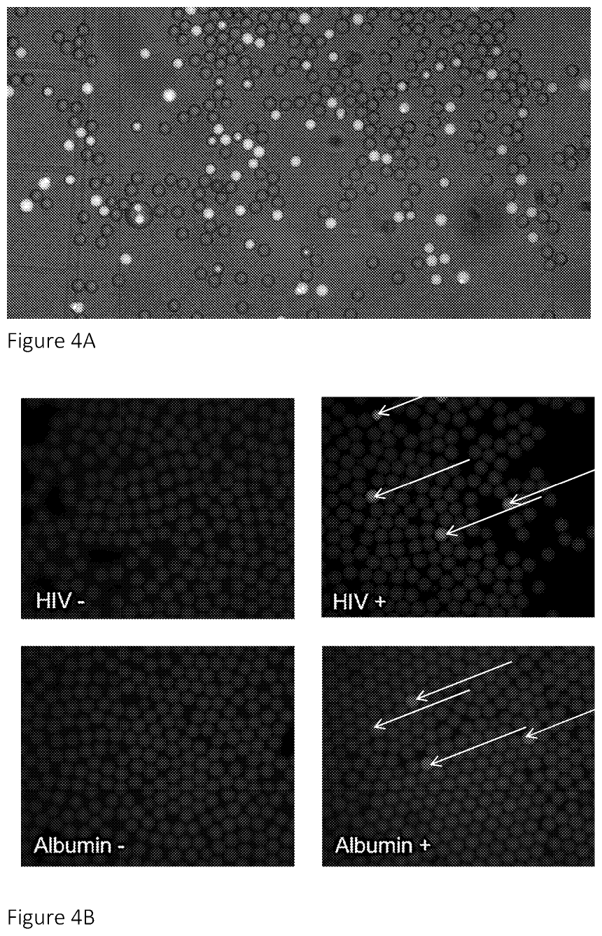

FIG. 4A—shows the result of PCR in WOs created on the microfluidic chip of FIG. 1a. Fluorescent droplets can be seen, the bright fluorescent droplets are in PCR positive WOs—that is WOs in which PCR occurred, and the dark droplets are PCR negative WOs where the PCR reaction was unsuccessful due to a lack of target DNA. Fluorescence was achieved by staining WOs with 200× SYBR Green I Nucleic Acid Stain post thermocycling.

FIG. 4B—shows the result of PCR in WOs created on the microfluidic chip of FIG. 1a. In this example, amplification of both the human target (Albumin) and the viral target (5′ LTR) can be seen. Droplets containing the target of interest are identifiable by the increased fluoresence intensity. The two panels on the left show representative droplets from a no-template control in-drop PCR, all droplets are of the same intensity as no amplification of the target has occurred. The two panels on the right show the same PCR reactions using a positive control template. Droplets containing the DNA fragment of interest exhibit a higher fluorescent signal due to amplification of the target within those droplets, these droplets are marked by an arrow.

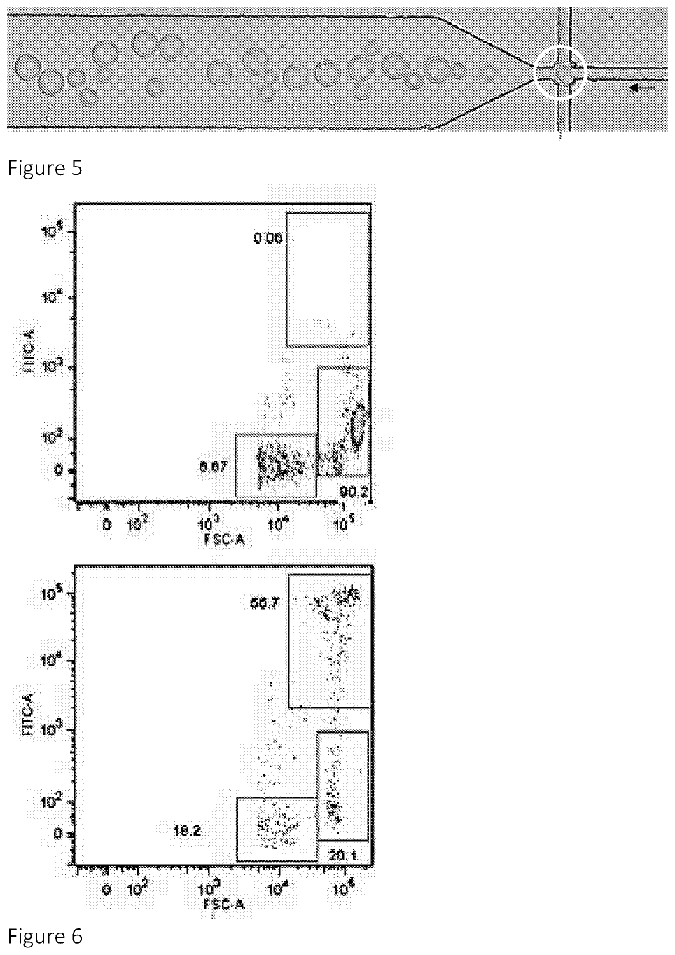

FIG. 5—shows the formation of a WOW (water in oil in water) double emulsion. The image shows WOWs leaving the flow focusing junction (circle). Flow direction is indicated by arrows (horizontal arrow=aqueous inlet, vertical arrow=Oil inlet).

FIG. 6—shows FACS gating of WOWs recovered from a microfluidic device. FIG. 6a is a plot showing representative FACS gating of PCR-negative WOWs (WOWs containing cells that do not contain latent HIV-1 DNA). Bottom left gate contains oil-in-water droplets (OWs) formed when a WO is not loaded into a WOW, bottom right gate contains PCR negative WOWs that are FITC^{low}, top right gate contains PCR positive WOWs (WOWs containing cells that contain latent HIV-1 DNA) that are FITC^{high} (this gate is empty in FIG. 6a) FIG. 6b is a plot showing representative FACS gating of PCR positive WOWs—the top right gate now contains PCT positive cells.

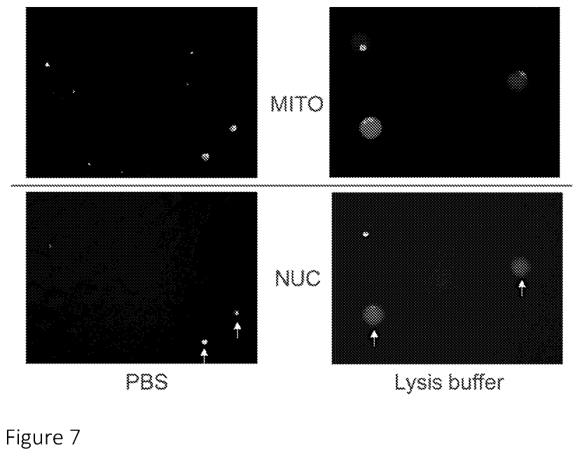

FIG. 7—provides evidence of in-drop cell lysis. The top left box shows cells (red) encapsulated in droplets stained with a mitochondrial stain in a PBS control, the top right box shows cell lysis in the presence of lysis buffer as evidenced by the fact that the mitochondrial stain has been able to migrate from within the cell to fill the droplet. The bottom left and right boxes show the same as the time but using a different stain, in this case for a nuclear stain (green).

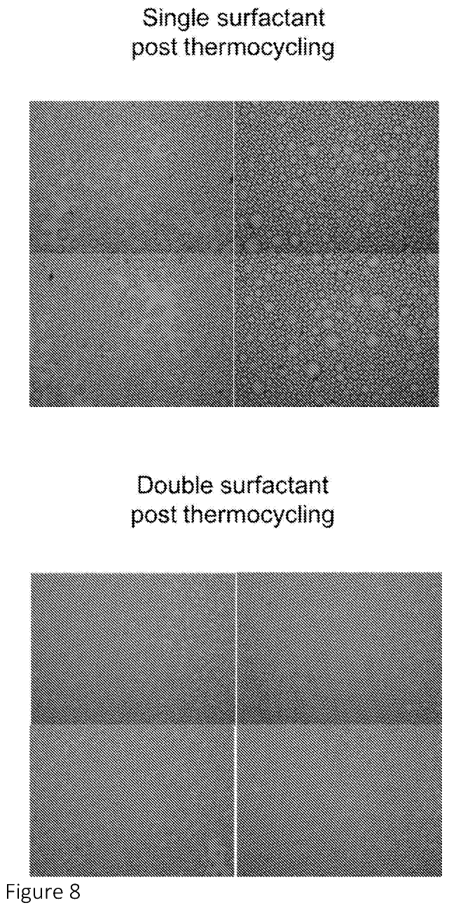

FIG. 8—demonstrates the ability of a non-ionic surfactant, such as octylphenoxy poly(ethyleneoxy)ethanol (Igepal) to stabilise the droplets. The four images on the left show droplets post thermocycling when only a single surfactant is used for droplet production. The four images on the right show droplets post thermocycling using the second surfactant (Igepal). The droplets remain monodisperse despite heating to 95° C.

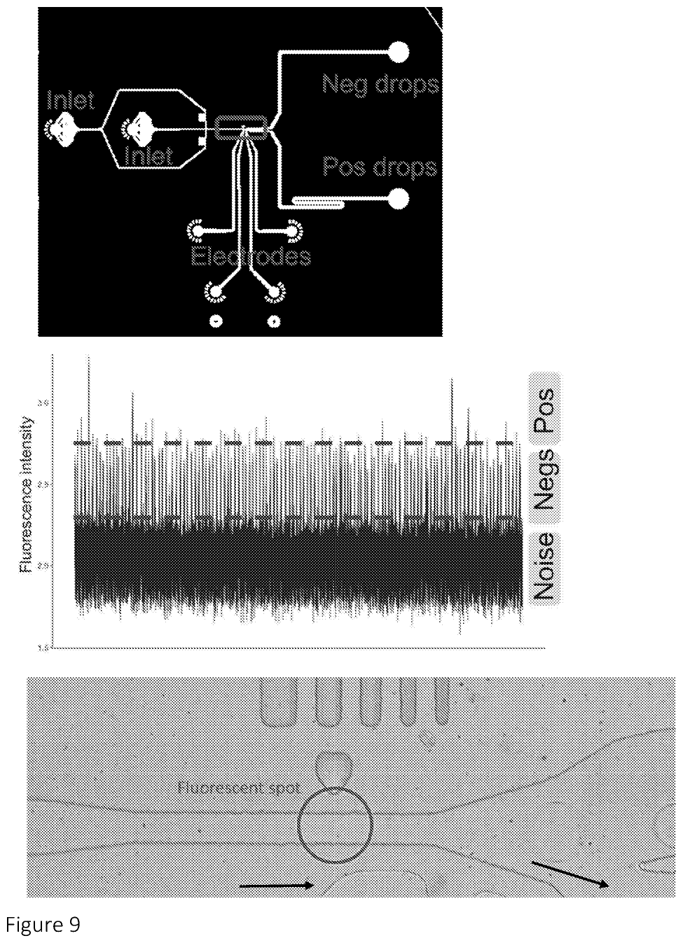

FIG. 9—demonstrates how the droplets can be sorted on the microfluidic device. The top image is a schematic of a sorting device. The middle image is a plot showing fluorescence detection by a photomultiplier tube (PMT) as droplets pass through it. The dashed lines are thresholds between the signal noise from the PMT, the amplitude of the negative droplet intensity and the amplitude of the positive droplet intensity. Droplets that emit a fluorescent intensity above a set threshold trigger an electrode (green arrow) to produce a dielectrophoretic pulse that deflects the target droplet towards a separate sort channel (blue arrow).

### MATERIALS AND METHODS

### Silicon Wafer Fabrication

Microfluidic devices for use in the method of the invention were constructed by first spin-coating a silicon wafer with SU8-2035 at successively greater speeds as per the table below.

| Step | Speed (rpm) | Time | Acceleration (rpms-1) |
|---|---|---|---|
| 1 | 500 | 15 seconds | 100 |
| 2 | 1300 | 38 seconds | 100 |
| 3 | 0 | 10 minutes | 0 |

SU8-coated wafers are then thermocycled on a hotplate according to the following table.

| Step | Temperature (° C.) | Time (minutes) |
|---|---|---|
| 1 | 65 | 5 |
| 2 | Ambient | 3 |
| 3 | 95 | 20 |
| 4 | Ambient | 3 |

Post thermocycling the silicon wafer was covered with the patterned photomask and exposed to UV light for 30 seconds. Areas of SU8 polymer exposed to UV at this stage become cross-linked and resistant to chemical degradation at later stages in the protocol. UV exposed wafers were subsequently thermocycled once more according to the following table.

| Step | Temperature (° C.) | Time (minutes) |
|---|---|---|
| 1 | 65 | 5 |
| 2 | Ambient | 3 |
| 3 | 95 | 10 |
| 4 | Ambient | 3 |

At this stage, a latent image was visible on the silicon wafer.

To develop the image, the wafer was immersed in developer for 9 minutes (whilst shaking), removed from the developer and then sprayed with developer for 10 seconds. Following a rinse with Isopropanol and drying with N2, the wafer was placed on a hotplate at 150° C. for 20 minutes.

### Silicone Elastomer Chip Production

Sylgard 184 (Dow Corning) base was mixed with Sylgard 184 curing agent at a ratio of 10:1 and centrifuged briefly to remove bubbles from the mixture. The elastomer mixture was slowly poured over elastomer from the silicon wafer and placed channel side up on a clean work surface. Using a scrap piece of elastomer underneath the chip, the inlet and outlet holes were pierced with a 1 mm biopsy punch. The newly pierced chip was cleaned and a microscope slide using an ethanol spray bottle then blow dry with N2. The microscope slide and chip (channels facing up) were placed into a plasma cleaner and exposed to oxygen plasma for one minute. The chip was then removed from the plasma cleaner immediately and gently pressed (channels facing down) on to the slide. The slide and chip are now bonded. For water-in-oil droplet (WO) chips it is necessary to make the chip hydrophobic, this was achieved using hexamethyldisilazane (HMDS). The bonded chip was placed into a sealed container with 500u1 of HMDS and left for 4 hours. HMDS penetrated the elastomer and the vapour coated the channels of the chip leaving a single molecule hydrophobic coating on the surface.

### Making Single Cell Water in Oil Droplets (‘WOs’)

Cells were pre-stained with antibodies of interest and washed with 1×PBS. Inlet syringes were loaded as per the table below.

| Syringe name | Contents | Flow rate (ul/min) |
|---|---|---|
| Inlet syringe 1 | Cell suspension | 1 |
| Inlet syringe 2 | 2× DNA amplification | 1 |
| | reagents or lysis buffer | |
| Inlet syringe 3 | HFE7500 with surfactant | 4 |

All inlet syringes were attached to syringe pumps and allowed to flow into the WO chip/device (FIG. 1a) with the rates in the above table. Droplets were formed at the flow focusing junction of the chip. A high speed camera (capable of capturing >10,000 frames per second) was used to monitor cell loading efficiency, some minor adjustments to flow rate may be required for optimal cell loading (FIG. 2). WOs were collected in a 200 ul PCR tube layered underneath 100 ul mineral oil. Cell lysis was performed off chip by incubating at 55° C. for 30 minutes followed by a 10 minute 95° C. enzyme inactivation step. PCR amplification reagents were introduced into the droplet either by picoinjection or droplet merger. Dependent upon the DNA amplification method employed either the WOs were theromocycled (PCR) or proceeded straight to WOW production. If using a PCR method, the denaturation temperature was reduced to 93° C. or lower during thermocycling. (FIG. 3). After thermocycling the WOs were left in the thermocycler at 4° C. to allow them to stabilise. Post thermocycling, droplets which fluoresce contain a cell (FIG. 4)

### Making Water-in-Oil-in-Water (‘WOW’) Double Emulsion

Conventional flow cytometers are not able to analyse droplets in an organic background and require an aqueous background. It is necessary therefore to create a double emulsion of WOWs (FIG. 5). This is achieved by using a WOW chip/microfluidic device (FIG. 1b) with inlet syringes loaded as described in the table below.

| Syringe name | Contents | Flow rate (ul/min) |
|---|---|---|
| Inlet syringe 1 | WO suspension | 0.5 |
| Inlet syringe 2 | Water + 0.1% Tween-20 | 4 |

Using the flow rates detailed in the table above it is possible to encapsulate each WO into a new WOW where the two aqueous phases inside and outside the droplet are separated by a thin oil shell. WOWs are then collected directly in to FACS tubes and stored at 4° C.

### Droplet Analysis and Sorting

### Sorting by Conventional Flow Cytometry

WOWs can be treated the same as other cells when being analysed on a flow cytometer. Analysis parameters can include forward scatter, side scatter and any detectable antibodies that were stained for prior to encapsulation. In order to provide an accurate quantification primers targeting both the HIV-1 LTR and the human albumin gene were incorporated into the DNA amplification reactions. During flow cytometry a cell is interrogated by fluorescent laser light for both of these targets (FIG. 6). The possible combinations of these targets are detailed below.

| | Albumin | HIV-1 |
|---|---|---|
| Empty droplet | − | − |
| Uninfected cell loaded | + | − |
| HIV-1 infected cell loaded | + | + |

Droplets that do not match the +/+ phenotype of a HIV-1 infected cell are counted but not sorted. +/+ cells are counted and sorted into individual wells for further downstream analysis. The data collected at this stage allows a basic quantification of reservoir to be made by determining the proportion of cells that also contain integrated HIV-1 DNA.

### On-Chip Dielectrophoretic Sorting

WOs can be sorted using a single-use PDMS sorting microfluidic device as detailed in FIG. 9. Droplets are injected into the microfluidic device where they are spaced using oil and driven past a fluorescent spot. A photomultiplier tube captures the fluorescence exhibited by each droplet and by using a microcontroller (attached to a function generator, voltage amplifier and electrode) can generate a dielectrophoretic field to deflect the droplet of interest towards a sort channel on the device. These droplets can be collected into a suitable vessel for downstream processing

### Post Sorting Analysis

Post sorting, droplets are processed for either next-generation DNA sequencing, RNA-sequencing or target enrichment using a customised Agilent™ SureSelect™ assay to increase specificity of capture. This is possible due to the DNA/RNA still being viable within the sorted +/+ droplets.

### CD4 T Cell Enrichment

Prior to cell analysis and droplet formation a population of cells maybe enriched for CD4 T-cells. CD4 enrichment may be achieved by using a commercially available immunomagnetic negative selection antibody capture assay such as the STEMCELL Technologies EasySep kit. Unwanted cells are targeted for removal with Tetrameric Antibody Complexes recognizing non-CD4+T cells and dextran-coated magnetic particles. Labeled cells are then separated using an EasySep™ magnet without the use of columns.

### PCR Primers and Cycling Conditions

To detect for human albumin DNA in a cell an albumin PCR mastermix containing 2× Lightcycler 480 Probes Master Mix (Roche, Welwyn Garden City, UK), 200 nM

Probe (FAM—CCT GTC ATG CCC ACA CAA ATC TCT CC—BHQ-1), 250 nM Albumin F (GCT GTC ATC TCT TGT GGG CTG T) and 250 nM Albumin R (AAA CTC ATG GGA GCT GCT GGT T) (Eurofins MWG Operon, Ebersberg, Germany) was used.

To detect for HIV DNA in a cell an HIV-1 mastermix was used, which contained 500 nM Probe (FAM—AGT RGT GTG TGC CCG TCT GTT G—BHQ-1), 500 nM LTR OS (GRA ACC CAC TGC TTA ASS CTC AA) and 500 nM LTR AS (TGT TCG GGC GCC ACT GCT AGA GA) (Eurofins MWG Operon) and 2× LightCycler 480 probe Master Mix, in a total volume of 25 ul.

Both qPCR amplications were performed using the following program: one cycle of 95° C. for 10 min; 45 cycles of 95° C. for 15 s and 60° C. for 1 min.

1. A method of identifying a cell latently infected with HIV, wherein the method comprises:
(a) providing a sample of cells;
(b) encapsulating individual cells in droplets;
(c) screening for the presence of HIV derived DNA in the genomic DNA of encapsulated cells; and
(d) identifying, and optionally isolating, cells containing latent HIV derived DNA.

2. The method of claim 1 wherein the droplets are water in oil droplets.

3. The method of claim 1, wherein the sample of cells is processed to produce a solution of single cells.

4. The method of claim 1, wherein the sample of cells is obtained from a biological sample obtained from a subject.

5. The method of claim 1, wherein the sample of cells is derived from a tissue biopsy, a blood sample, a sample of any other bodily fluid sample, or any sample from which single cells can be derived.

6. The method of claim 1, wherein the sample of cells are subjected to an enrichment step to isolate and enrich for CD4⁺ T-cells in the sample.

7. The method of claim 1, wherein the sample of cells are obtained from a human.

8. The method of claim 1, wherein the sample of cells are obtained from a subject that has previously been diagnosed with an HIV infection, and is optionally taking antiretroviral therapy.

9. The method of claim 1, wherein the results of step (d) are used in identifying latent HIV-1 or HIV-2 infection in CD4 T cells or other potential host cells.

10. The method of claim 1, further comprising the step of lysing the cell membrane and nuclear membrane of an encapsulated cell prior to screening for the presence of HIV derived DNA.

11. The method of claim 1, wherein the cells are encapsulated in a droplet with the reagents needed to detect the presence of a specific DNA sequence in the genomic DNA of the cell, or wherein the reagents needed to detect the presence of a specific DNA sequence in the genomic DNA of the cell are added to the droplet after the cell has been lysed.

12. The method of claim 11 wherein the reagents comprise a PCR/isothermal mix comprising the primers and enzymes necessary to amplify and detect a specific DNA sequence in the genomic DNA of the encapsulated cell.

13. The method of claim 1, further comprising the step of screening to allow the number of droplets containing a cell to be determined, irrespective of whether the cell carries latent HIV DNA.

14. The method of claim 13 wherein the presence of a cell is determined by:
i) a labelled antibody or probe against a phenotypic marker, or
ii) by performing a PCR or an isothermal DNA amplification reaction for a human genomic target using labelled primers.

15. The method of claim 13 further comprising the step of determining what percentage of cells in a population carries latent HIV.

16. The method of claim 1, further comprising the step of determining whether the HIV is replication competent.

17. The method of claim 1, further comprising the step of isolating cells found to carry a particular genomic marker from the rest of the cell population for further analysis.

18. The method of claim 1 wherein the method is carried out using a microfluidic device, and cells in the sample are encapsulated in droplets on the microfluidic device, wherein the cells are encapsulated in water in oil droplets.

19-20. (canceled)

21. The method of claim 1 further including the step of using the cells identified to provide an accurate measure of the size and nature of the reservoir of latent HIV in a subject.

22. A method of determining the size of the reservoir of cells latently infected with HIV-1 in a subject comprising:
(i) providing a sample of CD4+ cells from a subject;
(ii) encapsulating individual cells in droplets;
(iii) lysing the cell membrane and the nuclear membrane of the cell in the droplet;
(iv) introducing reagents necessary to amplify a specific HIV-1 target;
(v) subjecting the cells to conditions to allow amplification of HIV-1 derived DNA if present in the genome an encapsulated cell;
(vi) screening the droplets to identify any in which DNA amplification has occurred;
(vii) quantifying the number of cells that contain HIV-1 derived DNA;
(viii) quantifying the total number of cells; and
(ix) using the total number of cells and the number of cells that contain HIV-1 derived DNA to determine the size of the reservoir of cells latently infected with HIV-1 in a subject.

23. The method of claim 22 further comprising the step of isolating individual cells/droplets that contain HIV-1 derived DNA and analysing the HIV-1 derived DNA to determine if it is replication-competent.

24. A method of determining the replication competent viral load of a subject diagnosed with an HIV infection, wherein the method comprises the steps of:
i) obtaining a blood sample from the subject;
ii) enriching for CD4⁺ T cells in the sample;
iii) encapsulating individual CD4⁺ cells in a water in oil droplet on a microfluidic device;
iv) lysing the cell and nuclear membrane of encapsulated cells;
v) introducing DNA amplification reagents to the droplets containing lysed cells;
vi) placing the droplets on the device in conditions that permit amplification of any HIV DNA in a cell in a droplet;
vii) counting and/or isolating droplets in which amplification of HIV DNA has occurred; and optionally
viii) analysing the genomic DNA in the isolated droplets to determine if the HIV DNA is replication competent.

25. A method of using genotypic and phenotypic analysis to identify rare cells in a population, wherein the method comprises:
(a) providing a sample of cells;
(b) screening for the presence of cells expressing a particular phenotypic marker;
(c) screening for the presence of cells expressing a particular genotypic marker; and
(d) identifying, and optionally isolating, cells with one or more of the following properties:
cells expressing the particular phenotypic marker;
cells expressing the particular genotypic marker;
cells expressing the particular phenotypic and genotypic marker; and
cells expressing the neither the phenotypic or the genotypic marker.
